(19) United States
(12) Patent Application Publication
(10) Pub. No.: US 2026/0276639 A1
(43) Pub. Date: Sep. 17, 2026

(54) KAT6A AS A PREDICTIVE BIOMARKER FOR TREATMENT OF BREAST CANCER WITH A CDK4 INHIBITOR AND AN ANTIESTROGEN AND METHODS OF TREATMENT THEREOF

(71) Applicant: PFIZER INC., NEW YORK, NY (US)

(72) Inventors: Shibing DENG, San Diego, CA (US); Li LIU, San Diego, CA (US); Yuan LIU, San Diego, CA (US)

(73) Assignee: PFIZER INC., NEW YORK, NY (US)

(21) Appl. No.: 19/470,726

(22) PCT Filed: Mar. 27, 2024
(86) PCT No.: PCT/IB2024/052985

§ 371 Date: Sep. 29, 2025

Related U.S. Application Data
(60) Provisional application No. 63/455,970, filed on Mar. 30, 2023.

(51) Int. Cl. G01N 33/57515; A61K 31/4196; A61K 31/506; A61K 31/519; A61K 31/565; C12Q 1/6886

(52) U.S. Cl. CPC G01N 33/57515; A61K 31/4196; A61K 31/506; A61K 31/519; A61K 31/565; C12Q 1/6886; C12Q 2600/158

(57) ABSTRACT

This invention relates to a method of selecting a subject having a cancer for treatment, comprising: i) determining a KAT6A level from a biological sample of the cancer from the subject; ii) selecting the subject for treatment with a cyclin-dependent kinase 4 (CDK4) inhibitor as a single agent or in combination with an antiestrogen, based on the KAT6A level, wherein the KAT6A level is determined to be low, and methods of treatment thereof. It is shown that lower than median KAT6A mRNA levels in metastatic tumor samples of patients with breast cancer qualify as a predictive biomarker for the response to a combination therapy with palbociclib and fulvestrant.

### BACKGROUND OF THE INVENTION

The present invention relates to methods of selecting patients for cancer treatment with a CDK4 inhibitor. In particular, this invention relates to methods of selecting a patient, based on a KAT6A level of the patient's cancer, wherein the KAT6A level is determined to be low, for treatment with a CDK4 inhibitor as a single agent or in combination with an antiestrogen, such as an aromatase inhibitor, a selective estrogen receptor degrader (SERD) or a selective estrogen receptor modulator (SERM).

KAT6A and its paralog KAT6B are lysine acetyltransferases from the MYST family with H3 acetyltransferase activity. KAT6A and KAT6B are highly homologous to each other at the sequence level (AA identity 60% & similarity 66%) and display similar properties in in vitro H3K23 acetylation and functions. KAT6A and KAT6B genes have both been identified as top-ranking targets amplified in different solid tumor types, which may drive oncogenic process in cancer. However due to their distinct expression patterns, KAT6A and KAT6B may have different regulations and functions in vivo.

KAT6A regulates gene transcription, cell cycle, senescence and cell differentiation (HUANG, F., et al., “Regulation of KAT6 Acetyltransferases and Their Roles in Cell Cycle Progression, Stem Cell Maintenance, and Human Disease,” Molecular and Cellular Biology, 2016, 1900-1907, vol. 36, no. 14). KAT6A deregulation has been shown to associate with tumorigenesis with its increased or altered activity associated with several cancers including glioblastoma, estrogen receptor (ER)-driven breast cancer, androgen receptor (AR)-driven prostate cancer and lymphoma (YANG, X-J., “MOZ and MORF acetyltransferases: Molecular interaction, animal development and human disease,” 2015, Biochimica et Biophysica Acta, 1818-1826, vol. 1853, no. 8).

The KAT6A gene is amplified and overexpressed in a subset of breast cancers and may positively regulate ER expression. Identification of KAT6A gene amplification as part of the 8p11-p12 amplicon in breast cancer has indicated its potential role as an oncogene in luminal breast cancers (TURNER-IVEY, B., et al., “KAT6A, a Chromatin Modifier from the 8p11-p12 Amplicon is a Candidate Oncogene in Luminal Breast Cancer,” 2014, Neoplasia, 644-655, vol. 16, no. 8). Increased KAT6A activity in KAT6A gene amplified and overexpressed ER+breast cancer cells may drive cell proliferation through its transcriptional regulation of ER pathway including ESR1 expression (YU, L., et al., “Identification of MYST3 as a novel epigenetic activator of ERα frequently amplified in breast cancer,” 2017, Oncogene, 2910-2918, vol. 36, no. 20).

Allelic variants of KAT6A gene (in most cases loss of function) are associated with an autosomal dominant form of cognitive disability (KENNEDY, J., et al., “KAT6A Syndrome: genotype-phenotype correlation in 76 patients with pathogenic KAT6A variants,” 2019, Genetics in Medicine, 850-860, vol. 21, no. 4). However, in cancer, KAT6A alterations are mostly gene amplification, gene overexpression, and recurrent gene translocation fusions, although other mutations have been also observed in various cancers. KAT6A gene is in 8p11-12 amplicon which includes other genes such as POLB, IKBKB, NSD3, FGFR1, etc. KAT6A gene amplifications are more frequently detected in invasive breast carcinoma (~9%, n=2912), prostate cancer (~10%, n=537) especially in CRPC (29%, n=44) and neuroendocrine prostate cancer (18%, n=44), esophageal squamous carcinoma (12%, n=95), lung squamous cancer (8%, n=25), bladder cancer (8%, n=73), uterine carcinoma (13%, n=79), and ovarian cancer (5%, n=584) (CERAMI, E., et al., “The cBio Cancer Genomics Portal: An Open Platform for Exploring Multidimensional Cancer Genomics Data,” Cancer Discovery, 2012, 401-404, vol. 2, no. 5; GAO, J., et al., “Integrative Analysis of Complex Cancer Genomics and Clinical Profiles Using the cBioPortal,” Science Signaling, 2013, 1-19, vol. 6., no. 269).

In addition to gene amplification, KAT6A overexpression may also contribute to oncogenesis. Although it was reported to be commonly seen in AML, breast, esophageal, ovarian, lung and stomach cancer, the frequency of KAT6A overexpression in cancer is still largely unknown due to the limitation of data sets reported.

Most cancer drugs are effective in some patients, but not in others. This may be due to genetic variation among tumors and may be observed even among tumors within the same patient. Variable patient response is particularly pronounced with respect to targeted therapeutics. Therefore, the full potential of targeted therapies may not be realized without suitable tests for determining which patients will benefit from which drugs. According to the National Institutes of Health (NIH), the term “biomarker” is defined as “a characteristic that is objectively measured and evaluated as an indicator of normal biologic or pathogenic processes or pharmacological response to a therapeutic intervention.”

There are three distinct types of cancer biomarkers: (1) prognostic biomarkers, (2) predictive biomarkers, and (3) pharmacodynamic biomarkers. A prognostic biomarker is used to classify a cancer, e.g., a solid tumor, according to aggressiveness, i.e., rate of growth and/or metastasis, and refractiveness to treatment. This is sometimes called distinguishing “good outcome” tumors from “poor outcome” tumors. A predictive biomarker is used to assess the probability that a particular patient will benefit from treatment with a particular drug. For example, patients with breast cancer in which the ERBB2 (HER2 or NEU) gene is amplified are likely to benefit from treatment with trastuzumab (HERCEPTIN©), whereas patients without ERBB2 gene amplification are unlikely to benefit from treatment with trastuzumab. A pharmacodynamic biomarker is an indication of the effect(s) of a drug on a patient while the patient is taking the drug. Accordingly, pharmacodynamic biomarkers often are used to guide dosage level and dosing frequency, during the early stages of clinical development of a new drug. For a discussion of cancer biomarkers, see, e.g., SAWYERS, C., “The cancer biomarker problem,” 2008, Nature, 548-552, vol. 452, no. 7187.

As such, there is a need to select which patients might respond to treatment with a CDK4 inhibitor alone or in combination. Therefore, there is a need for diagnostic methods based on predictive biomarkers that may be used to identify cancer patients that are likely (or unlikely) to respond to treatment with a CDK4 inhibitor as a single agent or in combination.

### SUMMARY OF THE INVENTION

The present invention provides, in part, methods of selecting patients and identifying cancers for treatment with a CDK4 inhibitor, and methods of treatment thereof.

According to a first embodiment of the invention, there is provided a method of selecting a subject having a cancer for treatment, comprising:
i) determining a KAT6A level from a biological sample of the cancer from the subject; and
ii) selecting the subject for treatment with one of:
   a) a cyclin-dependent kinase 4 (CDK4) inhibitor; and
   b) a cyclin-dependent kinase 4 (CDK4) inhibitor and an antiestrogen, based on the KAT6A level, wherein the KAT6A level is determined to be low.

According to a second embodiment of the invention, there is provided a method of treating a cancer in a subject, comprising:
i) determining a KAT6A level from a biological sample of the cancer from the subject;
ii) selecting the subject for treatment with one of:
   a) a cyclin-dependent kinase 4 (CDK4) inhibitor; and
   b) a cyclin-dependent kinase 4 (CDK4) inhibitor and an antiestrogen, and
iii) administering to the selected subject an amount of
   a) the cyclin-dependent kinase 4 (CDK4) inhibitor; or
   b) the cyclin-dependent kinase 4 (CDK4) inhibitor and the antiestrogen, wherein the amounts are effective in treating the cancer.

Described below are embodiments of the invention, where for convenience Embodiment 1 (E1) is identical to the method of selecting a subject having a cancer provided above, and Embodiment 2 (E2) is identical to the method of treating a cancer in a subject provided above.

It is to be understood that both the foregoing general description and the following detailed description are exemplary and explanatory only and are not restrictive of the invention, as claimed.

### BRIEF DESCRIPTION OF THE DRAWINGS

FIG. 1A shows the concordance between common gene expression levels detected by a KAT6A/B spiked PIP assay and RNAseq results from the same control tumor cells and ER+ HER2− metastatic tumor samples.

FIG. 1B shows the linear correlations for KAT6A gene expression detection by a KAT6A/B spiked PIP assay and RNAseq transcriptomic analysis.

FIG. 1C shows the linear correlations for KAT6A gene expression detection by a KAT6A/B spiked PIP assay and RNAseq transcriptomic analysis.

FIG. 2 is a forest plot of progression-free survival by subgroups in the PALOMA-3 study by mRNA expression level of KAT6A, where a high KAT6A mRNA expression level is defined as greater than median and a low KAT6A mRNA expression level is defined as less than or equal to median (>median: high, ≤median: low). Abbreviations include: 95% interval, 95% confidential interval; HR, hazard ratio; m, months; N, number; FUL, fulvestrant; PAL, pablociclib; Palbo, palbociclib+fulvestrant arm; and Ful, placebo+fulvestrant arm. Patient Subgroup Datasets include: ITT, Intent-To-Treat Patient Cohort; ITT_PriorChemo, patients in the ITT Patient Cohort who received prior chemotherapy; BM_All, Biomarker All Patient Cohort; BM_All_KAT6A low, patients in the BM_All Patient Cohort with a low KAT6A mRNA expression level; BM_All_KAT6A high, patients in the Biomarker All Patient Cohort with a high KAT6A mRNA expression level; BM_PriorChemo; patients in the Biomarker All Patient Cohort who received prior chemotherapy; BM_PriorChemo_KAT6A low, patients in the Biomarker All Patient Cohort who received prior chemotherapy and have a low KAT6A mRNA expression level; BM_PriorChemo_KAT6A high, patients in the Biomarker All Patient Cohort who received prior chemotherapy with a high KAT6A mRNA expression level.

FIG. 3 shows a Kaplan-Meier plot of progression-free survival by subgroups in the Biomarker All Patient Cohort of the PALOMA-3 study by mRNA expression level of KAT6A, where a high KAT6A mRNA expression level is defined as greater than median and a low KAT6A mRNA expression level is defined as less than or equal to median (>median: high, ≤median: low). Abbreviations include: FUL, fulvestrant; PAL, pablociclib. Time is in months.

FIG. 4 shows a Kaplan-Meier plot of progression-free survival by subgroups of patients with prior chemotherapy in the Biomarker All Patient Cohort of the PALOMA-3 study by mRNA expression level of KAT6A, where a high KAT6A mRNA expression level is defined as greater than median and a low KAT6A mRNA expression level is defined as less than or equal to median (>median: high, ≤median: low). Abbreviations include: FUL, fulvestrant; PAL, pablociclib. Time is in months.

FIG. 5 show KAT6A expression level in patient subgroups by clinical characteristics in the PALOMA-3 study. Patient characteristics include: ALL, Biomarker All Patient Cohort; PrioChemo_NonSens. to HRi, prior chemotherapy and were not sensitive to hormone therapy; NonSens to HRi, not sensitive to hormone therapy; DFI<=24m, disease-free intervals that were less than or equal to 24 months; Primary tumors, primary tumors were submitted for biomarker analysis; Non Visceral, non-viseral diseases; PrioChemo, prior chemotherapy; Post Menopausal, post menopause at the study entry; PrioChemo_Sens. to HRi, prior chemotherapy and were sensitive to hormone therapy; DFI>24m, disease-free intervals that were more than 24 months; Sens. to HRi, sensitive to hormone therapy; Visceral.Mets, viseral metastatic diseases; Metastatic, metastatic tumors were submitted for biomarker analysis; Pre/Peri Menopausal, not post menopause at the study entry; Chemo Naive, no prior chemotherapy

### DETAILED DESCRIPTION OF THE INVENTION

The present invention may be understood more readily by reference to the following detailed description of the embodiments and preferred embodiments of the invention. It is to be understood that the terminology used herein is for the purpose of describing specific embodiments only and is not intended to be limiting. It is further to be understood that unless specifically defined herein, the terminology used herein is to be given its traditional meaning as known in the relevant art.
E1 A method of selecting a subject having a cancer for treatment, as defined above.
E2 A method of treating a cancer in a subject, as defined above.
E3 The method of embodiment 1 or embodiment 2, wherein the CDK4 inhibitor is a CDK4 selective inhibitor or a CDK4/6 inhibitor.
E4 The method of any one of embodiments 1 to 3, wherein the CDK4 inhibitor is a CDK4 selective inhibitor.
E5 The method of embodiment 4, wherein the CDK4 selective inhibitor is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a pharmaceutically acceptable salt thereof.
E6 The method of any one of embodiments 1 to 3, wherein the CDK4 inhibitor is a CDK4/6 inhibitor.
E7 The method of embodiment 6, wherein the CDK4/6 inhibitor is abemaciclib, ribociclib or palbociclib, or a pharmaceutically acceptable salt thereof.
E8 The method of embodiment 7, wherein the CDK4/6 inhibitor is palbociclib, or a pharmaceutically acceptable salt thereof.
E9 The method of any one of embodiments 1 to 8, wherein the antiestrogen is an aromatase inhibitor, a selective estrogen receptor degrader (SERD), or a selective estrogen receptor modulator (SERM).
E10 The method of embodiment 9, wherein the antiestrogen is an aromatase inhibitor.
E11 The method of embodiment 9, wherein the antiestrogen is a selective estrogen receptor degrader (SERD).
E12 The method of embodiment 9, wherein the antiestrogen is a selective estrogen receptor modulator (SERM).
E13 The method of embodiment 9, wherein the antiestrogen is fulvestrant or letrozole.
E14 The method of embodiment 13, wherein the antiestrogen is fulvestrant.
E15 The method of embodiment 13, wherein the antiestrogen is letrozole.
E16 The method of any one of embodiments 1 to 15, wherein the low KAT6A level is determined based on KAT6A mRNA expression, KAT6A protein expression, KAT6A DNA gene amplification, or KAT6A DNA gene copy numbers.
E17 The method of embodiment 16, wherein the low KAT6A level is determined based on KAT6A mRNA expression.
E18 The method of embodiment 17, wherein the low KAT6A level is greater than a reference value from normal tissue using RNA sequencing, nanostring technology, or quantitative PCR or RNA in situ hybridization (ISH) technology.
E19 The method of embodiment 17 or embodiment 18, wherein the low KAT6A level is less than or equal to about 9.3 log 2 counts per million; about 9.2 log 2 counts per million; about 9.1 log 2 counts per million; or about 9 log 2 counts per million.
E20 The method of embodiment 16, wherein the low KAT6A level is determined based on KAT6A protein expression.
E21 The method of embodiment 20, wherein the low KAT6A level is less than or equal to a reference value from normal tissue using IHC assay.
E22 The method of embodiment 16, wherein the low KAT6A level is determined based on KAT6A DNA gene amplification.
E23 The method of embodiment 22, wherein the low KAT6A level is no DNA gene amplification.
E24 The method of embodiment 16, wherein the low KAT6A level is determined based on KAT6A DNA gene copy numbers.
E25 The method of embodiment 24, wherein the low KAT6A level is less than or equal to 2.
E26 The method of any one of embodiments 1 to 16, wherein step i) of embodiment 1 is determined by an assay that measures KAT6A mRNA expression.
E27 The method of any one of embodiments 1 to 16, wherein step i) of embodiment 1 is determined by an assay that measures KAT6A protein expression.
E28 The method of any one of embodiments 1 to 16, wherein step i) of embodiment 1 is determined by measuring KAT6A DNA gene amplification.
E29 The method of any one of embodiments 1 to 16, wherein step i) of embodiment 1 is determined by measuring KAT6A gene copy numbers.
E30 The method of any one of embodiments 26, 28 and 29, wherein step i) of embodiment 1 is performed by next generation sequencing.
E31 The method of any one of embodiments 1 to 30, wherein the biological sample is blood, serum, cells, or tissue.
E32 The method of embodiment 31, wherein the biological sample is cells or tissue.
E33 The method of any one of embodiments 1 to 32, wherein the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.
E34 The method of embodiment 33, wherein the cancer is breast cancer, lung cancer, prostate cancer, pancreatic cancer, or ovarian cancer.
E35 The method of embodiment 34, wherein the cancer is breast cancer, lung cancer, or prostate cancer.
E36 The method of embodiment 35, wherein the cancer is breast cancer.
E37 The method of embodiment 36, wherein the breast cancer is hormone receptor positive (HR+) breast cancer.
E38 The method of embodiment 37, wherein the hormone receptor positive (HR+) breast cancer is selected from the group consisting of progesterone receptor positive (PR+) breast cancer and estrogen receptor positive (ER+) breast cancer.
E39 The method of embodiment 38, wherein the hormone receptor positive (HR+) breast cancer is progesterone receptor positive (PR+) breast cancer.
E40 The method of embodiment 38, wherein the hormone receptor positive (HR+) breast cancer is estrogen receptor positive (ER+) breast cancer.
E41 The method of embodiment 38, wherein the estrogen receptor positive (ER+) breast cancer is human epidermal growth factor receptor 2 negative (HER2−).
E42 The method of embodiment 38, wherein the estrogen receptor positive (ER+) breast cancer is human epidermal growth factor receptor 2 positive (HER2+).
E43 The method of any one of embodiments 1 to 42, wherein the subject is human.

Each of the embodiments of the present invention described herein may be combined with one or more other embodiments of the present invention described herein which is not inconsistent with the embodiment(s) with which it is combined. In addition, each of the embodiments below describing the invention envisions within its scope the pharmaceutically acceptable salts of the compound of the invention.

As used herein, the singular form “a”, “an”, and “the” include plural references unless indicated otherwise.

As used herein, the term “about” when used to modify a numerically defined parameter (e.g., a KAT6A level) means that the parameter may vary by as much as 10% below or above the stated numerical value for that parameter. For example, a KAT6A level of about 9 means 9±10%, i.e., it may vary between 8.1 and 9.9.

Cyclin-dependent kinases (CDKs) and related serine/threonine kinases are important cellular enzymes that perform essential functions in regulating cell division and proliferation.

CDK inhibitors include Pan-CDK inhibitors that target a broad spectrum of CDKs or selective CDK inhibitors that target specific CDK(s).

Cyclin-dependent kinases (CDKs) and related serine/threonine protein kinases are important cellular enzymes that perform essential functions in regulating eukaryotic cell division and proliferation. The CDK catalytic units are activated by regulatory subunits known as cyclins.

At least sixteen mammalian cyclins have been identified (Johnson DG, Walker CL. Cyclins and Cell Cycle Checkpoints. Annu. Rev. Pharmacol. Toxicol. (1999) 39:295-312). Cyclin B/CDK1, cyclin A/CDK2, cyclin E/CDK2, cyclin D/CDK4, cyclin D/CDK6, and likely other heterodynes are important regulators of cell cycle progression. Additional functions of cyclin/CDK heterodynes include regulation of transcription, DNA repair, differentiation and apoptosis (Morgan DO, Cyclin-dependent kinases: engines, clocks, and microprocessors. Annu. Rev. Cell. Dev. Biol. (1997) 13:261-291).

CDK inhibitors have been demonstrated to be useful in treating cancer. Increased activity or temporally abnormal activation of cyclin-dependent kinases has been shown to result in the development of human tumors, and human tumor development is commonly associated with alterations in either the CDK proteins themselves or their regulators (Cordon-Cardo C. Mutations of cell cycle regulators: biological and clinical implications for human neoplasia. Am. J. Pathol. (1995) 147:545-560; Karp JE, Broder S. Molecular foundations of cancer: new targets for intervention. Nat. Med. (1995) 1:309-320; Hall M, Peters G. Genetic alterations of cyclins, cyclin-dependent kinases, and Cdk inhibitors in human cancer. Adv. Cancer Res. (1996) 68:67-108).

CDK4 and CDK6 are important regulators of cell cycle progression at the G1-S checkpoint, which are controlled by D-type cyclins and INK4 endogenous CDK inhibitors, such as p16^{INK4a} (CDKN2A). Dysregulation of the cyclin D-CDK4/6-INK4-retinoblastoma (Rb) pathway has been reported to be associated with development of endocrine therapy resistance. Furthermore, CDK4 has been identified as the singular oncogenic driver in many breast cancers and emerging data suggest that cyclin D3-CDK6 inhibition may be linked to hematologic toxicity, suggesting a role for CDK4 selective inhibitors.

Clinical trials for the CDK4/6 inhibitors palbociclib, ribociclib and abemaciclib are ongoing for breast and other cancers, as single agents or in combination with other therapeutics. The use of CDK4/6 inhibitors in combination with endocrine therapy has demonstrated significant efficacy in the treatment of hormone receptor (HR)-positive, human epidermal growth factor 2 (HER2)-negative advanced or metastatic breast cancers, and CDK4/6 inhibitors, including palbociclib, ribociclib and abemaciclib, have been approved in combination with endocrine therapy in a first- or second-line setting. Palbociclib, ribociclib and abemaciclib have been approved for treatment of hormone receptor (HR)-positive, human epidermal growth factor receptor 2 (HER2)-negative advanced or metastatic breast cancer in combination with aromatase inhibitors, such as letrozole, in a first line setting and with fulvestrant in second or later lines of therapy in certain patients. (O'Leary et al. Treating cancer with selective CDK4/6 inhibitors. Nature Reviews (2016) 13:417-430).

As used herein, a “CDK4 inhibitor” includes a CDK4 selective inhibitor and a CDK4/6 inhibitor. “CDK4 selective inhibitor” refers to compounds that inhibit the kinase activity of CDK4 to a greater extent than any other CDKs. “CDK4/6 inhibitor” refers to compounds that inhibit the kinase activity of CDK 4 and 6. CDK4 selective inhibitors are disclosed in International Publication No. WO 2019/207463. Examples of CDK4/6 inhibitors include, but are not limited to, abemaciclib, ribociclib and palbociclib. Additional examples of CDK4/6 inhibitors include lerociclib (also known as G1T38) and trilaciclib (also known as GT1128).

In an embodiment, CDK4 selective inhibitors of the present invention include 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (also known as as “PF-07220060”), or a pharmaceutically acceptable salt thereof. PF-07220060 is a potent and selective inhibitor of CDK4, having the structure:

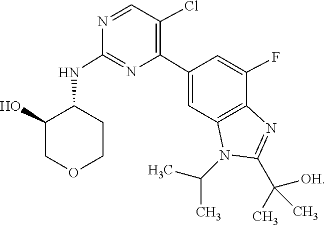 [SMILES: CC(C)n1c(C(C)(C)O)nc2c(F)cc(-c3nc(N[C@@H]4CCOC[C@H]4O)ncc3Cl)cc21]

PF-07220060 and pharmaceutically acceptable salts thereof, are disclosed in International Publication No. WO 2019/207463, U.S. Pat. Nos. 10,766,884, 11,220,494, and US Patent Publication US 2022/0089580; International Publication No. WO 2022/058871, and International Publication No. WO 2023/100070. The contents of which are incorporated herein by reference in their entirety. Unless indicated otherwise, all references herein to PF-07220060 include references to salts, solvates, hydrates and complexes thereof, and to solvates, hydrates and complexes of salts thereof, including polymorphs, stereoisomers, and isotopically labelled versions thereof.

In an embodiment, a CDK4/6 inhibitor of the present invention includes palbociclib, or a pharmaceutically acceptable salt thereof. Palbociclib, or 6-acetyl-8-cyclopentyl-5-methyl-2-(5-piperazin-1-yl-pyridin-2-ylamino)-8H-pyrido[2,3-d]pyrimidin-7-one (also known as “PD-0332991” and referred to herein as “palbo” or “PAL”) is a potent and selective inhibitor of CDK4 and CDK6, having the structure:

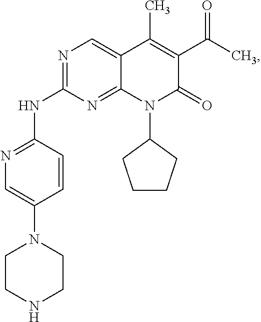 [SMILES: CC(=O)c1c(C)c2cnc(Nc3ccc(N4CCNCC4)cn3)nc2n(C2CCCC2)c1=O]

Palbociclib is described in WHO Drug Information, Vol. 27, No. 2, page 172 (2013). Palbociclib and pharmaceutically acceptable salts thereof are disclosed in International Publication No. WO 2003/062236 and U.S. Pat. Nos. 6,936,612, 7,456,168 and RE47,739; International Publication No. WO 2005/005426 and U.S. Pat. Nos. 7,345,171 and 7,863,278; International Publication No. WO 2008/032157 and U.S. Pat. No. 7,781,583; and International Publication No. WO 2014/128588. The contents of each of the foregoing references are incorporated herein by reference in their entirety.

As used herein, “endocrine therapy” or “hormone therapy” means an aromatase inhibitor, a selective estrogen receptor degrader (SERD), or a selective estrogen receptor modulator (SERM). In certain embodiments, endocrine therapy includes fulvestrant, tamoxifen, toremifene, anastrozole, exemestane, or letrozole.

The term “antiestrogen” as used herein refers to a class of drugs that prevent estrogens like estradiol from mediating the biological effects in the body. Antiestrogens act by blocking the estrogen receptor (ER) and/or inhibiting or suppressing estrogen production. In an embodiment, an antiestrogen is an aromatase inhibitor, a selective estrogen receptor degrader (SERD) or a selective estrogen receptor modulator (SERM). Examples of an aromatase inhibitor include, but are not limited to, anastrozole. Examples of a SERD include, but are not limited to, fulvestrant. Additional SERDs include elacestrant (RAD-1901, Radius Health), SAR439859 (Sanofi), RG6171 (Roche), AZD9833 (AstraZeneca), AZD9496 (AstraZeneca), rintodestrant (G1 Therapeutics), ZN-c5 (Zentalis), LSZ102 (Novartis), D-0502 (Inventisbio), LY3484356 (Lilly), and SHR9549 (Jiansu Hengrui Medicine). Examples of a SERM include, but are not limited to, tamoxifen, clomifene and raloxifene. Additional SERMS include toremifene, lasofoxifene, bazedoxifene and afimoxifene.

In an embodiment, the aromatase inhibitor includes letrozole, exemestane, and anastrozole. In an embodiment, the SERM includes tamoxifen, clomifene and raloxifene.

In an embodiment, an antiestrogen of the present invention includes fulvestrant and letrozole. In an embodiment, an antiestrogen of the present invention includes fulvestrant. In an embodiment, an antiestrogen of the present invention includes letrozole.

Another embodiment relates to the pharmaceutically acceptable salts of the compounds described herein. Pharmaceutically acceptable salts of the compounds described herein include the acid addition and base addition salts thereof.

Another embodiment also relates to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of the compounds described herein. Suitable base addition salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine and zinc salts.

The compounds described herein that are basic in nature are capable of forming a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds of the compounds described herein that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

Selecting Subjects for Treatment based on a KAT6A Level

The present invention relates to the use of KAT6A as a predictive biomarker for patient-selection.

As used herein, the terms, “subject”, “participant” and “patient,” are used interchangeably, to refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used therein, a biological sample is used to refer to blood, plasma, cell and/or tissue samples collected from a subject, participant or patient.

As used herein, a “KAT6A level” is a level of KAT6A measured by a suitable assay in a biological sample of a subject.

A KAT6A level may be determined, or measured, by any of a number of assays and methodologies known to one of ordinary skill in the art, including by not limited to, KAT6A mRNA expression, KAT6A protein expression, KAT6A DNA gene amplification, and KAT6A DNA gene copy numbers.

A KAT6A level may be determined, or measured, from tumor tissue or tumor cells by a KAT6A mRNA expression assay. A KAT6A mRNA expression assay may be used to determine whether the KAT6A level in the tissue is high or low. Examples of suitable KAT6A mRNA expression assays, include but are not limited to:
HTG EdgeSeq KAT6A/B spiked PIP assay described in Example 1.
RNA sequencing for whole exome or targeted panel including KAT6A such as ACE exome RNAseq assay or ImmuneID NeXT exome RNAseq assay provided by Personalis Inc (Menlo Park, CA).
Nanostring technology such as tumor signaling 360 panel including KAT6A assay (Seattle, WA).
Quantitative PCR to quantify KAT6A RNA expression levels from tumor tissues or cells, such as real time PCR provided by AppliedBiosystems (Foster City, CA). Using KAT6A sequence-specific primers, the number of molecules of KAT6A RNA sequence may be determined by measuring the amount of amplified product at each stage during the PCR cycle.
RNA in situ hybridization (ISH) technology such as RNAscope ISH assay provided by Advanced Cell Diagnostics, Inc. (Hayward, CA). RNA ISH assay uses nucleotide probes specific for KAT6A for in situ hybridization on tumor tissues or cells to visualize and spatially detect KAT6A RNA expression molecules at the single cell level, whilst maintaining the spatial tissue microenvironment.

A KAT6A level may be determined, or measured from tissue or cells, by KAT6A protein expression using antibodies directed against any peptide or protein fragment specific for KAT6A by Immunohistochemical (IHC) staining assay, ELISA assay and western blot analysis. KAT6A protein levels can also be measured by mass spectrometry method based on KAT6A amino acid sequences and molecule weight. KAT6A protein expression may be used to determine whether the KAT6A level in the tissue or cells is high or low.

A KAT6A level may be determined, or measured, from tumor tissue or cells, blood serum or plasma by KAT6A DNA gene amplification and KAT6A DNA copy numbers. KAT6A DNA gene amplification and KAT6A DNA copy numbers may be used to determine whether the KAT6A level in the tissue or cells, serum or plasma is high or low. Examples of suitable methods of measuring KAT6A DNA gene amplification and KAT6A DNA copy numbers, include but are not limited to:
DNA next generation sequencing (NGS) for whole genome or targeted panel including KAT6A such as ImmuneID NeXT or ACE Extended Cancer Panel for DNA provided by Personalis Inc (Menlo Park, CA). KAT6A gene amplification and copy numbers higher or lower than normal may be quantified.
DNA next generation sequencing (NGS) detection for blood serum or plasma panel including KAT6A such as Guardant Omni provided by Guardant Health (Redwood City, CA). KAT6A gene amplification and copy numbers higher or lower than normal may be quantified.
Quantitative PCR such as real time PCR provided by AppliedBiosystems (Foster City, CA) to quantify KAT6A DNA copy numbers from tumor tissues or cells or cell free serum or plasma from peruperal blood. Using KAT6A sequence-specific primers, the number of copies of KAT6A DNA sequence may be determined by measuring the amount of amplified product at each stage during the PCR cycle.
Fluorescence in situ hybridization (abbreviated FISH) test such as the FISH Tag detection provided by Thermal Fishers (Waltham, MA). This test detects amplification of the KAT6A gene region (8p11.21) via fluorescence in situ hybridization in tumor tissue specimens. It detects and locates a DNA sequence specific for KAT6A on a chromosome. In this technique, the full set of chromosomes from subject is affixed to a glass slide and then exposed to a “probe”—a small piece of purified DNA tagged with a fluorescent dye. The fluorescently labeled probe finds and then binds to its matching sequence within the set of chromosomes. With the use of a special microscope, the chromosome and sub-chromosomal location and fluorescent intensity where the fluorescent probe bound may be seen.

A high KAT6A level and a low KAT6A level, which may also be described as a positive for high KAT6A level or a negative for high KAT6A level, respectively, may be defined using a cutoff value such as a KAT6A expression level median/mean value or any cutoff value which is defined by its association or predictiveness of the cancer treatment benefit (Hui, J. et al, Optimal Biomarker Cutof Identification and Validation, Statistics in Biosciences (2022) 14:352-362). KAT6A level “high” or “low” may be defined as a KAT6A level above or below a cut-off value based on a percentile, mean or median value of KAT6A mRNA expression, KAT6A protein expression, or KAT6A DNA gene copy number values from a group of cancer patients. For example, as detailed in Example 1 (the PALOMA-3 clinical study), a median KAT6A mRNA expression value of 9.27 log 2 counts per million was used as a cutoff value to define a high KAT6A level versus a low KAT6A level. A KAT6A level “high” or “low” may also be based on the presence or absence of KAT6A gene amplification. Any KAT6A biomarker score above the cutoff value may be defined as KAT6A high. Any KAT6A biomarker score below the cutoff value may be defined as KAT6A low. One of ordinary skill in the art would be able to determine whether a KAT6A value is high or low.

### Administration and Dosing

“Treat” or “treating” a cancer and/or a cancer-associated disease as used herein means to administer a monotherapy or combination therapy according to the present invention to a subject, participant or patient having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, or inhibiting the progress of the disorder or condition to which such term applies, or one or more symptoms of such disorder or condition. The term “treatment” or “therapy,” as used herein, unless otherwise indicated, refers to the act of treating as “treating” is defined immediately above. For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer may be measured in a number of ways (see, for example, W. A. Weber, J. Nucl. Med. 50:1S-10S (2009)).

As used herein, the terms, “subject”, “participant” and “patient,” are used interchangeably, to refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

An “amount” for use and for treating a subject refers to an amount that provides, in single or multiple doses, alone, or in combination with one or more other agents, a detectable response of any duration of time (transient, medium or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an agent, alone, or in combination with one or more other agents, effective for producing a desired therapeutic effect upon administration to a subject, for example, to stem the growth, or result in the shrinkage, of a cancerous tumor. In reference to the treatment of cancer, a therapeutically effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression.

As used herein, “ameliorate” refers to any reduction in the extent, severity, frequency, and/or likelihood of a symptom or clinical sign characteristic of a particular disease. “Symptom” refers to any subjective evidence of disease or of a subject's condition.

Administration of the compounds of the present invention may be effected by any method that enables delivery of the compounds to the site of action. These methods include oral routes, intraduodenal routes, parenteral injection (including intravenous, subcutaneous, intramuscular, intravascular or infusion), topical, and rectal administration. Each compound may be administered according to the same or different route of administration.

In a preferred embodiment, the daily dose of a CDK4 inhibitor or a pharmaceutically acceptable salt thereof, is administered orally.

A CDK4 inhibitor, or a pharmaceutically acceptable salt, may be present in a pharmaceutical composition which includes a pharmaceutically acceptable excipient. “Pharmaceutically acceptable excipient” refers to a component that may be included in the compositions described herein, is physiologically suitable for pharmaceutical use, and causes no significant adverse effects nor therapeutic effects to a subject. The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

The amount of a CDK4 inhibitor, or a pharmaceutically acceptable salt, in the pharmaceutical compositions may be any amounts disclosed herein.

The compounds of the method, use or combination of the present invention may be formulated prior to administration. The formulation will preferably be adapted to the particular mode of administration. These compounds may be formulated with pharmaceutically acceptable excipients as known in the art and administered in a wide variety of dosage forms as known in the art. Dosage unit forms or pharmaceutical compositions suitable for oral administration include, but are not limited to tablets, capsules, such as gelatin capsules, pills, powders, granules, aqueous and nonaqueous oral solutions and suspensions, packaged in containers adapted for subdivision into individual doses.

In another embodiment, the dosage of a compound or pharmaceutical composition described herein may vary within the range depending upon the dosage form employed and the route of administration utilized. In another embodiment, an amount of a compound or pharmaceutical composition described herein administered to a subject may be dependent upon factors known to a skilled artisan, including bioactivity and bioavailability of the compound (e.g., half-life and stability of the compound in the body), chemical properties of the compound (e.g., molecular weight, hydrophobility and solubility), route and frequency of administration, and the like. Further, it will be understood that the specific dose of a pharmaceutical composition comprising a compound as disclosed herein may depend on a variety of factors including physical condition of the subject (e.g., age, gender, weight), and medical history of the subject (e.g., medications being taken, health condition other diseases or disorders). The precise dose of a pharmaceutical composition administered to a subject may be determined by methods known to a skilled artisan such as a pharmacologist, or an anesthesiologist.

In an embodiment, palbociclib, or a pharmaceutically acceptable salt thereof, is administered at a daily dosage of about 125 mg once daily, about 100 mg once daily, about 75 mg once daily, about 50 mg daily, or about 25 mg daily. In an embodiment, which is the recommended starting dose, palbociclib, or a pharmaceutically acceptable salt thereof, is administered at a daily dosage of about 125 mg once a day. For example, palbociclib, or a pharmaceutically acceptable salt thereof, is administered at a dose of about 100 mg once daily, about 75 mg once daily, or about 50 mg once daily. In an embodiment, palbociclib, or a pharmaceutically acceptable salt thereof, is administered at a dose of about 100 mg once daily. In an embodiment, palbociclib, or a pharmaceutically acceptable salt thereof, is administered at a dose of about 75 mg once daily. In an embodiment, palbociclib, or a pharmaceutically acceptable salt thereof, is administered at a dose of about 50 mg once daily. Dosage amounts provided herein refer to the dose of the free base form of palbociclib, or are calculated as the free base equivalent of an administered palbociclib salt form. For example, a dosage or amount of palbociclib, such as 100 mg, 75 mg or 50 mg, refers to the free base equivalent.

In an embodiment, the CDK4 inhibitor, for example, 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a pharmaceutically acceptable salt thereof, is administered at a daily dosage of from about 1 mg to about 1000 mg per day. In another embodiment, the CDK4 inhibitor is administered at a daily dosage from about 10 mg to about 1000 mg per day. In another embodiment, the CDK4 inhibitor is administered at a dosage of from about 25 mg to about 900 mg per day. In another embodiment, the CDK4 inhibitor is administered at a dosage of from about 50 mg to about 800 mg per day. In another embodiment the CDK4 inhibitor is administered at dosages of about: 1, 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 260, 270, 275, 280, 290, 300, 325, 350, 375, 400, 425, 450, 475 or 500 mg on a QD, twice a day (BID), three times a day (TID) or four times a day (QID) schedule. In certain embodiments, the CDK4 inhibitor is administered at a dosage of about 50 mg QD, about 50 mg BID, about 75 mg QD, about 75 mg BID, about 200 mg QD, about 200 mg BID, about 300 mg QD, about 300 mg BID, about 400 mg QD, about 400 mg BID, or about 500 mg QD.

In an embodiment, 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a pharmaceutically acceptable salt thereof, is administered once or twice daily to comprise a complete cycle of 28 days. Repetition of the 28-day cycles is continued during treatment with the combination of the present invention.

epetition of the administration or dosing regimens may be conducted as necessary to achieve the desired reduction or diminution of cancer cells. A “continuous dosing schedule”, as used herein, is an administration or dosing regimen without dose interruptions, e.g., without days off treatment. Repetition of 28-day treatment cycles without dose interruptions between the treatment cycles is an example of a continuous dosing schedule. In an embodiment, the compounds of the combination of the present invention may be administered in a continuous dosing schedule. In an embodiment, the compounds of the combination of the present invention may be administered concurrently in a continuous dosing schedule.

In an embodiment, 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered once daily to comprise a complete cycle of 28 days. Repetition of the 28-day cycles is continued during treatment with the combination of the present invention.

The standard recommended dosing regimen, which includes the standard dosing schedule, for palbociclib, or a pharmaceutically acceptable salt thereof, is administration once daily for 21 consecutive days followed by 7 days off treatment to comprise a complete cycle of 28 days. Repetition of the 28-day cycles is continued during treatment with the combination of the present invention.

The standard clinical dosing regimen, for palbociclib, or a pharmaceutically acceptable salt thereof, is administration of 125 mg once daily for 21 consecutive days followed by 7 days off treatment to comprise a complete cycle of 28 days. Repetition of the 28-day cycles is continued during treatment with the combination of the present invention.

In further embodiments of the invention, 2-methoxy-N-{4-methoxy-6-[(1H-pyrazol-1-yl)methyl]-1,2-benzoxazol-3-yl}benzene-1-sulfonamide, or a pharmaceutically acceptable salt thereof, is administered in combination with palbociclib and letrozole, where the palbociclib is administered at 125 mg orally, once daily for 21 days followed by 7 days off, and where the letrozole is administered at 2.5 mg orally, daily.

The invention also relates to a kit comprising the therapeutic agents of the combination of the present invention and written instructions for administration of the therapeutic agents. In one embodiment, the written instructions elaborate and qualify the modes of administration of the therapeutic agents, for example, for simultaneous or sequential administration of the therapeutic agents of the present invention. In one embodiment, the written instructions elaborate and qualify the modes of administration of the therapeutic agents, for example, by specifying the days of administration for each of the therapeutic agents during a 28 day cycle.

### Method of Treatment

In one embodiment, the disclosure provides a method of treating a cancer in a subject in need thereof, which includes administering to the subject an amount of a cyclin-dependent kinase 4 (CDK4) inhibitor as described herein, in subjects based on a KAT6A level determined from a biological sample of the cancer in the subject. In another embodiment, the disclosure also provides a method for treating cancer of a subject which includes administering to the subject an amount of a cyclin-dependent kinase 4 (CDK4) inhibitor as described herein in combination with an antiestrogen, based on a KAT6A level determined from a biological sample of the cancer in the subject.

The term “combination”, as used herein, unless otherwise indicated, means a fixed-dose combination or a combination of agents that is administered intermittently, concurrently or sequentially, according to the same or different route of administration and according to the same or different dosage schedules. As used herein, an “effective” or a “therapeutically effective” amount refers to an amount of an agent, compound, or composition that is of sufficient quantity to result in a decrease in severity of disease symptoms, an increase in frequency and duration of disease symptom-free periods, or a prevention of impairment or disability due to the disease affliction—either as a single dose or according to a multiple dose regimen, alone or in combination with other agents. One of ordinary skill in the art would be able to determine such amounts based on such factors as the patient's size, the severity of the patient's symptoms, and the particular combination, composition or route of administration selected. The patient or subject may be a human or non-human mammal in need of treatment. In one embodiment, the patient is human.

The term “locally advanced”, as used herein, as it relates to cancer, may or may not be treated with curative intent. The term “metastatic” as used herein, as it relates to cancer, cannot be treated with curative intent. Those skilled in the art will be able to recognize and diagnose locally advanced and metastatic cancer in a patient.

For convenience, certain well-known abbreviations, may be used herein, including: castration resistant prostate cancer (CRPC), estrogen receptor positive (ER+), human epidermal growth factor receptor 2 negative (HER2−), hormone receptor (HR), human epidermal growth factor receptor 2 positive (HER2+), non-small cell lung cancer (NSCLC) and progesterone receptor (PR).

In one embodiment, the cancer is selected from the group consisting of lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, head and neck cancer, or a combination of two or more of the foregoing cancers.

Another embodiment relates to methods of treating cancer in a patient. Another embodiment relates to the treatment of cancer in a patient comprising administering to the patient an amount of the compounds described herein that are effective in treating the cancer.

In one embodiment, the cancer is breast, lung, colon, brain, head and neck, prostate, stomach, pancreatic, ovarian, melanoma, endocrine, uterine, testicular, or bladder cancer.

In one embodiment, the cancer is breast, lung, prostate, pancreatic, or ovarian cancer.

In one embodiment, the cancer is breast, lung, or prostate cancer.

In one embodiment, the cancer is breast cancer.

In one embodiment, the breast cancer is HR+breast cancer.

In one embodiment, the HR+breast cancer is PR+ and/or ER+breast cancer.

In one embodiment, the breast cancer is PR+breast cancer.

In one embodiment, the breast cancer is ER+breast cancer.

In one embodiment, the breast cancer is ER+ HER2− breast cancer.

In one embodiment, the breast cancer is ER+ HER2+breast cancer.

In one embodiment, the breast cancer is locally advanced or metastatic ER+breast cancer.

In one embodiment, the breast cancer is locally advanced or metastatic ER+ HER2− breast cancer.

In one embodiment, the breast cancer is locally advanced or metastatic ER+ HER2+breast cancer.

In one embodiment, the lung cancer is non-small cell lung cancer.

In one embodiment, the lung cancer is locally advanced or metastatic non-small cell lung cancer.

In one embodiment, the prostate cancer is castration resistant prostate cancer.

In one embodiment, the prostate cancer is locally advanced or metastatic castration resistant prostate cancer.

Another embodiment relates to methods of treating solid tumors in a patient. Another embodiment relates to the treatment of solid tumors in a patient comprising administering to the patient an amount of the compounds described herein that are effective in treating the solid tumor.

In one embodiment, the solid tumor is breast, lung, colon, brain, head and neck, prostate, stomach, pancreatic, ovarian, melanoma, endocrine, uterine, testicular, or bladder.

In one embodiment, the solid tumor is breast, lung, prostate, pancreatic, or ovarian.

In one embodiment, the solid tumor is breast, lung, or prostate.

In one embodiment, the solid tumor is breast cancer, and in a further embodiment, the breast cancer is HR+breast cancer, and in a still further embodiment the HR+breast cancer is PR+ and/or ER+breast cancer ER+breast cancer.

In one embodiment, the solid tumor is breast cancer, and in a further embodiment, the breast cancer is ER+ HER2− breast cancer.

In one embodiment, the solid tumor is breast cancer, and in a further embodiment, the breast cancer is ER+ HER2+breast cancer.

In one embodiment, the solid tumor is breast cancer, and in a further embodiment, the breast cancer is locally advanced or metastatic ER+ HER2− breast cancer.

In one embodiment, the solid tumor is breast cancer, and in a further embodiment, the breast cancer is locally advanced or metastatic ER+ HER2+breast cancer.

In one embodiment, the solid tumor is lung cancer, and in a further embodiment the lung cancer is non-small cell lung cancer.

In one embodiment, the solid tumor is lung cancer, and in a further embodiment the lung cancer is locally advanced or metastatic non-small cell lung cancer.

In one embodiment, the solid tumor is prostate cancer, and in a further embodiment the prostate cancer is castration resistant prostate cancer.

In one embodiment, the solid tumor is prostate cancer, and in a further embodiment the prostate cancer is locally advanced or metastatic castration resistant prostate cancer.

Another embodiment relates to methods of treating hematologic tumors in a patient.

Another embodiment relates to the treatment of hematologic tumors in a patient comprising administering to the patient an amount of the compounds described herein that is effective in treating the hematologic tumor.

In one embodiment, the hematologic tumor is leukemia, lymphoma or multiple myeloma.

In one embodiment, the hematologic tumor is leukemia or lymphoma.

Another embodiment relates to methods of treating cancer in a patient with locally advanced or metastatic ER+ HER2− breast cancer, CRPC, or NSCLC whose disease progressed on or is intolerant to standard therapy.

Another embodiment relates to methods of treating cancer in a patient with locally advanced or metastatic ER+ HER2− breast cancer, CRPC, or NSCLC whose disease progressed on or is intolerant to standard therapy.

Another embodiment relates to methods of treating cancer in a patient with locally advanced or metastatic 2L+ ER+ HER2 breast cancer who has progressed after at least 1 prior line of treatment with an endocrine therapy and CDK4/6 inhibitor. In an embodiment thereof, the patient is administered a combination of a CDK4 inhibitor and fulvestrant.

Another embodiment relates to methods of treating cancer in a patient with locally advanced or metastatic 2L+ ER+ HER2 breast cancer who has progressed after at least 1 prior line of treatment with an endocrine therapy and CDK4/6 inhibitor. In an embodiment thereof, the patient is administered a combination of a CDK4 inhibitor with letrozole and palbociclib.

Another embodiment relates to methods of treating cancer in a patient with advanced or metastatic 2L+ ER+ HER2− breast cancer who has progressed after at least 1 prior line of CDK4/6 inhibitor and 1 line of endocrine therapy. In an embodiment thereof, the patient is administered a CDK4 inhibitor.

Another embodiment relates to methods of treating cancer in a patient with advanced or metastatic 2-4L fulvestrant-naive ER+ HER2− breast cancer whose disease has progressed after 1 line of a CDK4/6 inhibitor and 1 line of endocrine therapy and who must not have received more than 3 lines of systemic therapies in advanced or metastatic setting. In an embodiment thereof, the patient is administered a a CDK4 inhibitor and fulvestrant.

### EXAMPLES

### Example 1: KAT6A/B Spiked PIP HTG EdgeSeq Assay

Probes specific for KAT6A and KAT6B were designed and spiked into HTG EdgeSeq Precision Immuno-Oncology Panel (PIP, 1404 genes), referred to herein as the “KAT6A/B spiked PIP assay”. The KAT6A/B spiked PIP assay was established and validated at HTG Molecular Diagnostics, Inc., Tucson, Arizona (HTG Molecular) following HTG Molecular's standard assay development procedures (HTG Molecular Diagnostics, Inc., “HTG EdgeSeq System,” [White Paper], 24 Jul. 2017, Tucson, AZ). The samples used to develop the assay were ZR-75-1 (KAT6A gene amplified) and T47D (KAT6A overexpressed) formalin-fixed, paraffin-embedded (FFPE) cell pellets, and six ER+/HER2− metastatic human breast tumors. To validate the KAT6A/B spiked PIP assay, data generated from the KAT6A/B spiked PIP assay were compared to the gene expression levels detected by RNAseq Ace Cancer Transcriptome assay performed by Personalis (Personalis, Inc., Menlo Park, CA).

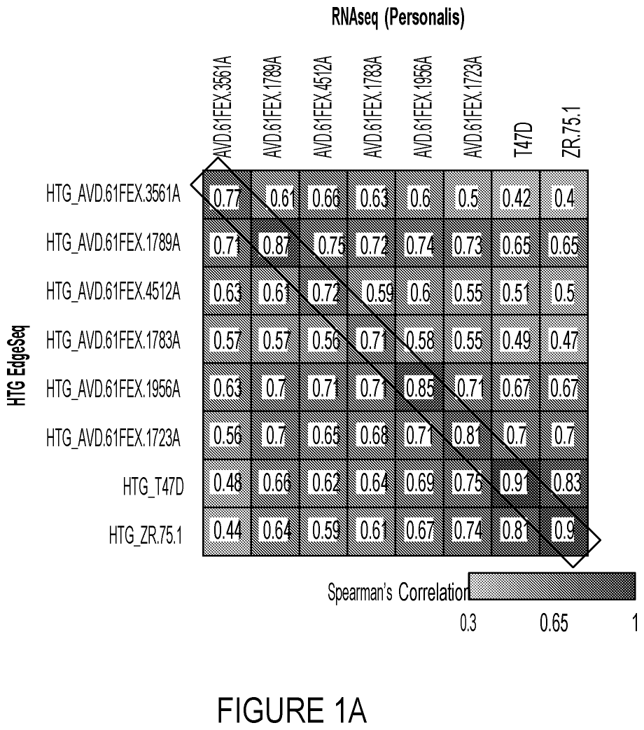

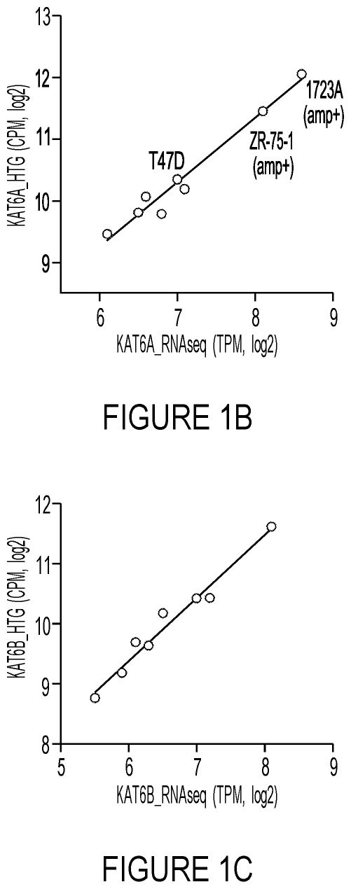

As shown in FIG. 1A, all common gene expression levels detected by the KAT6A/B spiked PIP assay showed an excellent concordance with the RNAseq results from the same control tumor cells and ER+ HER2− metastatic tumor samples with mean R²=0.82 (0.71-0.91). Excellent linear correlations were achieved for both KAT6A (slope=1.04, R²=0.97) (FIG. 1B) and KAT6B (slope=1.05, R²=0.97.) (FIG. 1C) gene expression detection by KAT6A/B spiked PIP assay and RNAseq transcriptomic analysis.

### Example 2: Gene Expression Analysis

A clinical trial entitled “multicenter, randomized, double-blind, placebo-controlled, phase 3 trial of fulvestrant (faslodex (registered)) with or without PD-0332991 (palbociclib)+/−goserelin in women with hormone receptor-positive, HER2-negative metastatic breast cancer whose disease progressed after prior endocrine therapy” (PALOMA-3, NCT01942135) was conducted.

Gene expression analysis in tumor samples from the PALOMA-3 study was performed using the KAT6A/B spiked PIP assay, as described in Example 1. The KAT6A/B spiked PIP assay, sample processing, library construction and sequencing were performed at HTG Molecular Diagnostics, Inc., (Tucson, AZ) in accordance with HTG EdgeSeq standard procedures and described in the HTG PIP product sheet. (HTG EdgeSeq Precision Immuno-Oncology Panel, “Enabling you to Decipher Complexities of the Tumor Microenvironment,” [White Paper], 28 May 2021, HTG Molecular Diagnostics, Inc., Tucson, AZ; SHI, Y., et al., “Evaluation of the EdgeSeq Precision Immuno-Oncology Panel for Gene Expression Profiling From Clinical Formalin-Fixed Paraffin-Embedded Tumor Specimens,” 2022, Frontiers in Cell and Developmental Biology, 1-11, vol. 10).

Tumor samples were collected from patients having HR+ HER2− advanced or metastatic breast cancer in the PALOMA-3 trial. The trial randomly assigned 521 endocrine pretreated patients (pts) including those who were prior exposed to chemotherapy with metastatic breast cancer to receive palbociclib (PAL) plus fulvestrant (FUL) or placebo (PBO) plus FUL in 2:1 ratio (“Intent-to-Treat Patient Cohort”). Pre- and peri-menopausal subjects were given an luteinizing hormone-releasing hormone (LHRH) agonist. Patients were treated with palbociclib (125 mg QD, 3/1 schedule) plus fulvestrant (500 mg intramuscular (IM) on Days 1 and 15 of Cycle 1, and then Day 1 of each subsequent 28-day cycle) or placebo (3/1 schedule QD) plus fulvestrant on the same schedule. The primary outcome was progression-free survival (PFS) as assessed by the investigator. Overall survival (OS) was one of the secondary endpoints.

Primary analysis was first conducted on KAT6A followed by a systematic panel-wide search. The association of gene expression with the effect of PAL+FUL versus PBO+FUL on PFS and OS was evaluated using Cox proportional hazards regression analysis, with gene expression as a continuous variable or dichotomized by median (expression>median: high, s median: low). Treatment by biomarker interaction effect was tested to identify potential predictive biomarkers. False discovery rate (FDR) was used to control multiplicity in exploratory analysis.

In the PALOMA-3 trial, the biomarker analyses were conducted in 214 patients (PAL+FUL arm, 137 pts; PBO+FUL arm, 77 pts) with available tumor samples at baseline (“Biomarker All Patient Cohort”), which included 165 patients with prior chemotherapy (PAL+FUL arm, 101 pts; PBO+FUL arm, 64 pts). 214 tumor samples were evaluable for analysis (111 archival primary samples [52%] and 103 metastatic biopsy samples [48%]). Of the evaluable samples, 137 (64%) were from the palbociclib+fulvestrant arm (70 primary samples and 67 metastatic samples), and 77 (36%) were from the placebo+fulvestrant arm (41 primary samples and 36 metastatic samples).

Baseline clinical and pathologic characteristics between the Biomarker All and Intent-to-Treat (ITT) Patient Cohorts are shown in Table 1. Median PFS between the Biomarker All and ITT Patient Cohorts are shown in Table 2.

TABLE 1
Demographics in Biomarker All Patient Cohort (214 patients)
versus Intent-to-Treat Patient Cohort (521 patients)

| | Overall, | | Gene Expression Profiling Data | | | | p- |
|---|---|---|---|---|---|---|---|
| Variable | N = 521¹ | | No, N = 307¹ | | Yes, N = 214¹ | | value² |
| Age | 57 | (49, 64) | 56 | (49, 64) | 58 | (49, 65) | 0.7 |
| Race | | | | | | | 0.3 |
| Asian | 105 | (20%) | 64 | (21%) | 41 | (19%) | |
| Black | 20 | (3.9%) | 11 | (3.6%) | 9 | (4.2%) | |
| Other | 9 | (1.7%) | 8 | (2.6%) | 1 | (0.5%) | |
| White | 385 | (74%) | 222 | (73%) | 163 | (76%) | |
| Treatment | | | | | | | 0.3 |
| Palbociclib + Fulvestrant | 347 | (67%) | 210 | (68%) | 137 | (64%) | |
| Placebo + Fulvestrant | 174 | (33%) | 97 | (32%) | 77 | (36%) | |
| Prior Hormone Therapy | 410 | (79%) | 244 | (79%) | 166 | (78%) | 0.6 |
| Prior Chemotherapy | | | | | | | 0.4 |
| No | 130 | (25%) | 81 | (26%) | 49 | (23%) | |
| Yes | 391 | (75%) | 226 | (74%) | 165 | (77%) | |
| Prior Chemotherapy Neoadjuvant | | | | | | | 0.2 |
| No | 307 | (59%) | 188 | (61%) | 119 | (56%) | |
| Yes | 214 | (41%) | 119 | (39%) | 95 | (44%) | |
| Prior Chemotherapy Adjuvant | | | | | | | 0.6 |
| No | 344 | (66%) | 200 | (65%) | 144 | (67%) | |
| Yes | 177 | (34%) | 107 | (35%) | 70 | (33%) | |
| Visceral Metastasis | 304 | (58%) | 195 | (64%) | 109 | (51%) | 0.004 |
| Menopausal Status | | | | | | | 0.003 |
| Post-Menopausal | 413 | (79%) | 230 | (75%) | 183 | (86%) | |
| Pre/Peri-Menopausal | 108 | (21%) | 77 | (25%) | 31 | (14%) | |
| ECOG PS³ | | | | | | | 0.3 |
| 0 | 322 | (62%) | 196 | (64%) | 126 | (59%) | |
| 1 | 199 | (38%) | 111 | (36%) | 88 | (41%) | |
| DFI24⁴ | | | | | | | 0.2 |
| 1 | 63 | (18%) | 32 | (16%) | 31 | (21%) | |
| 2 | 292 | (82%) | 174 | (84%) | 118 | (79%) | |
| Number of Prior Therapies | | | | | | | 0.8 |
| >=3 | 51 | (9.8%) | 32 | (10%) | 19 | (8.9%) | |
| 0 | 114 | (22%) | 65 | (21%) | 49 | (23%) | |
| 1 | 225 | (43%) | 130 | (42%) | 95 | (44%) | |
| 2 | 131 | (25%) | 80 | (26%) | 51 | (24%) | |

¹Data presented as No. (%); Age presented as median years (range from the 1st quartile to the 3rd quartile).
²Wilcoxon rank sum test; Fisher's exact test; Pearson's Chi-squared test
³Eastern Cooperative Oncology Group performance status (ECOG PS) describes a patient's level of functioning in terms of their ability to care for themself, daily activity, and physical ability
⁴Disease free interval for 24 months, 1 ≤ 24 months, 2 > 24 months. DFI was specified as length of time from primary treatment to disease relapse

TABLE 2
Median Progressive Free Survival in Biomarker All Patient Cohort (214
patients) versus Intent-to-Treat Patient Cohort (521 patients)

Patient Cohort

| | Intent-to-Treat | Biomarker All |
|---|---|---|
| Total number | 521 | 214 |

Palbociclib + Fulvestrant

| | | |
|---|---|---|
| Median PFS (months) | 11.2 | 12.0 |
| Number | 347 | 137 |

Fulvestrant alone (Placebo + Fulvestrant)

| | | |
|---|---|---|
| Median PFS (months) | 4.6 | 5.1 |
| Number | 174 | 77 |
| Hazard Ratio | (95% CI) = 0.50 | (95% CI) = 0.61 |
| | (0.40, 0.63) | (0.42, 0.87) |
| Log Rank | <0.0001 | 0.0056 |
| p Values | | |

As shown in Table 1 and Table 2, baseline clinical and pathologic characteristics and median PFS were similar between the Biomarker All and ITT Patient Cohorts. The Biomarker All Patient Cohort (patients with GEP data, n=214) had lower visceral metastases and higher percent of post-menopausal patients than the ITT Patient Cohort (n=521). The difference was relatively small (<10%).

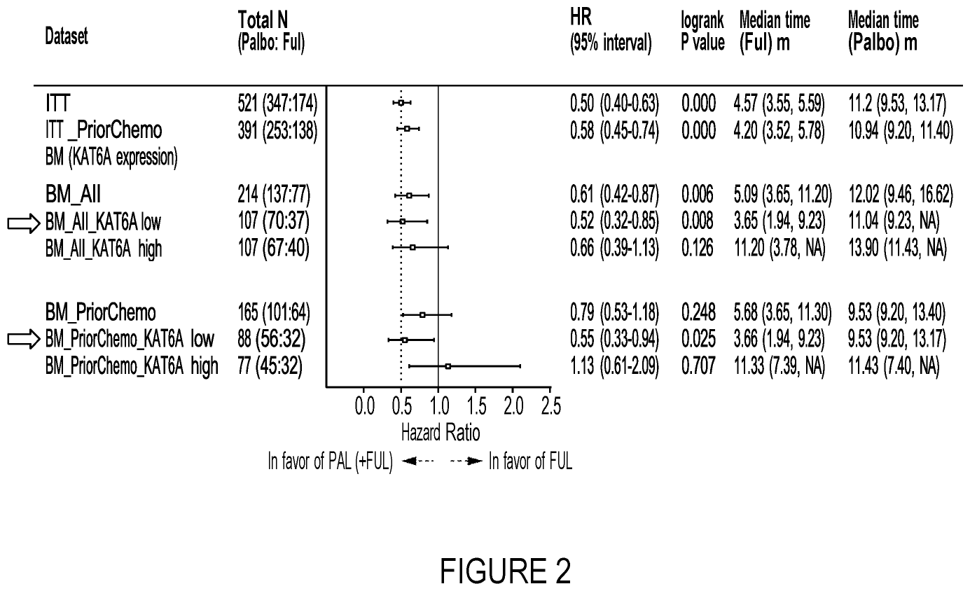

FIG. 2 shows a forest plot of progression-free survival by subgroups in the PALOMA-3 study by mRNA expression level of KAT6A, where a high KAT6A mRNA expression level is defined as greater than median and a low KAT6A mRNA expression level is defined as less than or equal to median (>median: high, ≤median: low). As shown in FIG. 2, lower KAT6A mRNA expression was associated with improved efficacy (lower Hazard Ratio) from the palbociclib+fulvestrant arm, especially in patients with prior chemotherapy.

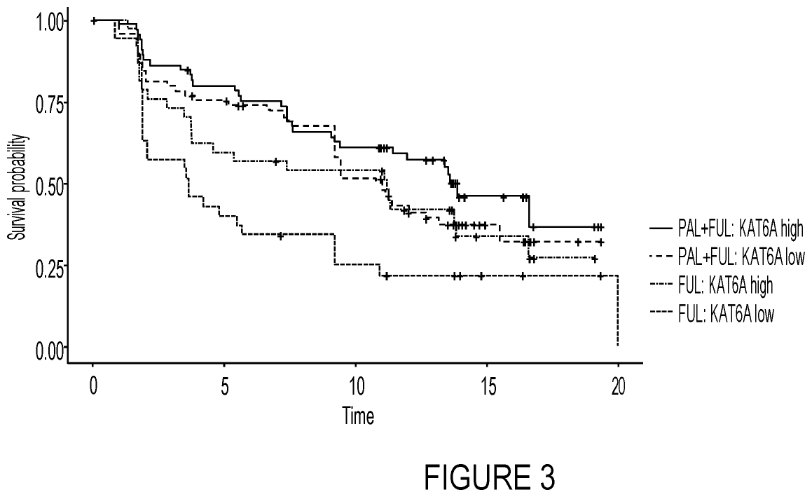

FIG. 3 shows a Kaplan-Meier plot of progression-free survival by subgroups in the Biomarker All Patient Cohort of the PALOMA-3 study by expression level of KAT6A, where a high KAT6A expression level is defined as greater than median and a low KAT6A expression level is defined as less than or equal to median (>median: high, ≤median: low). Table 3 shows supporting data for FIG. 3.

TABLE 3
Progression-Free Survival by Subgroups in the Biomarker All Patient
Cohort (n = 214) of the PALOMA-3 Study by Expression Level of KAT6A

| | KAT6A | | | | | |
|---|---|---|---|---|---|---|
| | Expression | | | | | |
| KAT6A | Value (log 2 | | | | | |
| Expression | counts per | Treatment | Number of | Number of | Median PFS | |
| Level | million) | Arms | Patients | Events | (95% CI) | |
| KAT6A low | 6.33-9.19 | PBO + FUL | 37 | 28 | 3.65 | (1.94, 9.23) |
| KAT6A high | 9.18-12.03 | PBO + FUL | 40 | 24 | 11.20 | (3.78, NA) |
| KAT6A low | 6.95-9.27 | PAL + FUL | 70 | 41 | 11.04 | (9.23, NA) |
| KAT6A high | 9.29-12.89 | PAL + FUL | 67 | 32 | 13.90 | (11.43, NA) |

As shown in FIG. 2, FIG. 3 and Table 3, in all patients with KAT6A expression data available, a favorable PFS for palbociclib+fulvestrant (versus placebo+fulvestrant) was observed in patients with low KAT6A gene expression (median PFS on palbociclib+fulvestrant was 11.04 months and placebo+fulvestrant was 3.65 months, Hazard Ratio (HR)=0.52, 95% Confidence Interval (CI) (0.32-0.85), log rank p=0.008) versus high KAT6A gene expression (median PFS on palbociclib+fulvestrant was 13.90 months and placebo+fulvestrant was 11.20 months, HR=0.66, 95% Cl (0.39-1.13), p=0.126). The interaction effect test for the benefit between the high and low KAT6A expression groups was not significant (interaction p=0.543). The results also showed that low KAT6A gene expression was associated with shorter PFS in the placebo+fulvestrant arm, but not in the palbociclib+fulvestrant arm (median PFS on placebo+fulvestrant was 3.65 months and palbociclib+fulvestrant was 11.04 months, Hazard Ratio (HR)=0.52, 95% Confidence Interval (CI) (0.32-0.85), log rank p=0.008). To summarize, in this biomarker cohort, a treatment benefit of adding palbociclib to fulvestrant was observed in patients with low KAT6A expression, but less in those with high KAT6A expression.

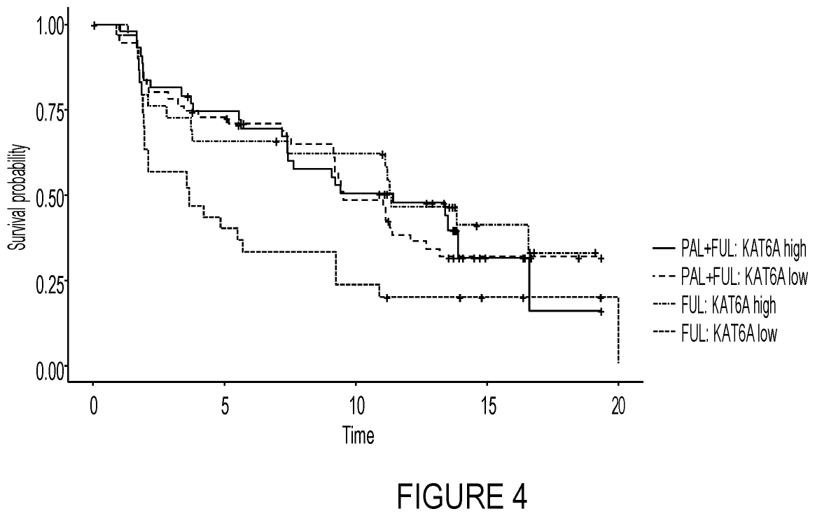

FIG. 4 shows a Kaplan-Meier plot of progression-free survival in subgroups of patients with prior chemotherapy in the Biomarker All Patient Cohort of the PALOMA-3 study by expression level of KAT6A, where a high KAT6A expression level is defined as greater than median and a low KAT6A expression level is defined as less than or equal to median (>median: high, ≤median: low). Table 4 shows supporting data for FIG. 4.

TABLE 4
Progression-Free Survival by Subgroups in the Biomarker
All Patient Cohort with Prior Chemotherapy (n =
165) of the PALOMA-3 Study by Expression Level of KAT6A

| | KAT6A | | | | | |
|---|---|---|---|---|---|---|
| | Expression | | | | | |
| KAT6A | Value (log2 | | | | | |
| Expression | counts per | Treatment | Number of | Number of | Median PFS | |
| Level | million) | Arms | Patients | Events | (95% CI) | |
| KAT6A low | 6.33-9.18 | PBO + FUL | 32 | 25 | 3.66 | (1.94, 9.23) |
| KAT6A high | 9.28-12.03 | PBO + FUL | 32 | 17 | 11.33 | (7.39, NA) |
| KAT6A low | 6.95-9.24 | PAL + FUL | 56 | 35 | 9.53 | (9.20, 13.17) |
| KAT6A high | 9.29-11.39 | PAL + FUL | 45 | 26 | 11.43 | (7.40, NA) |

As shown in FIG. 2, FIG. 4 and Table 4, KAT6A mRNA expression was more predictive in patients with prior chemotherapy. Favorable PFS for palbociclib+fulvestrant (versus placebo+fulvestrant) was observed in the patients who were prior exposed to chemotherapy with low KAT6A mRNA expression (median PFS on palbociclib+fulvestrant was 9.53 months and placebo+fulvestrant was 3.66 months, HR=0.55, 95% Cl (0.33-0.94), p=0.026), but not in those with high KAT6A expression (median PFS on palbociclib+fulvestrant was 11.43 months and placebo+fulvestrant was 11.33 months, HR=1.13, 95% Cl (0.61-2.09), p=0.71). The interaction effect test for the benefit between the high and low KAT6A expression groups was close to significant (interaction p=0.119). To summarize, in this biomarker cohort with prior chemotherapy, a treatment benefit of adding palbociclib to fulvestrant was observed in patients with low KAT6A expression, but not in those with high KAT6A expression.

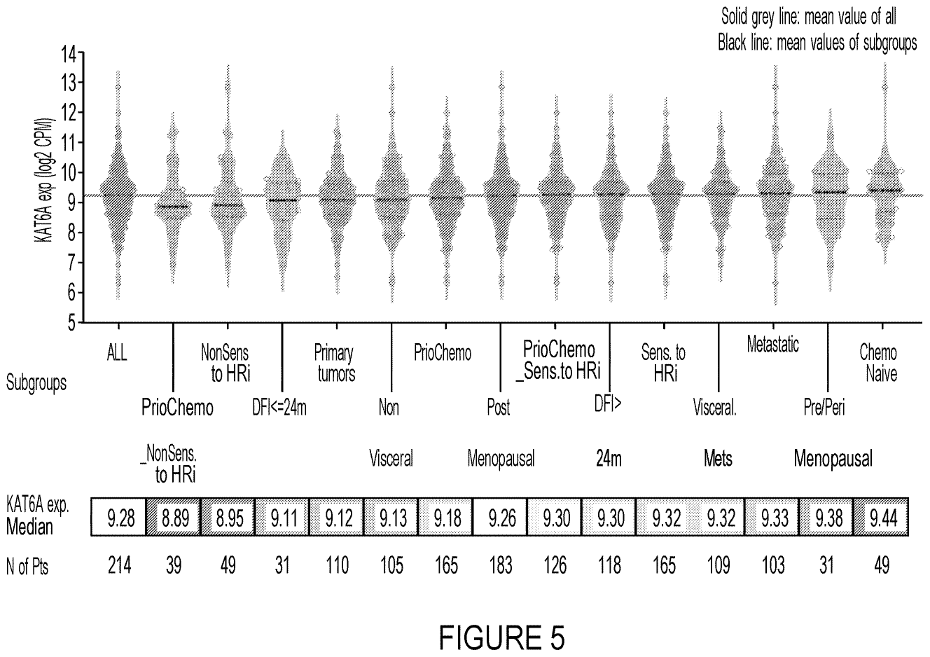

KAT6A was widely expressed in ER+ HER2− breast cancer tumors as shown in FIG. 5. Table 5 provides the descriptions of the clinical characteristics of the patient subgroups shown in FIG. 5. KAT6A expression levels were slightly lower in primary breast tumors than in metastatic lesions. KAT6A expression levels were slightly lower in patients with post menopausal status as compared to patients with pre/peri menopausal status. KAT6A expression levels were relatively lower in patients who were pre-treated with chemotherapy and/or not sensitive to hormone therapy as compared to patients who were sensitive to hormone therapy or chemotherapy naive with the order starting lowest: Prior chemo and not sensitive to HRi<not sensitive to HRi<Prior Chemo<Prior Chemo and sensitive to HRi<Chemo naive. KAT6A expression levels were slightly lower in patients who had disease free intervals less than or equal to 24 months as compared to patients who had disease free intervals of more than 24 months. KAT6A expression levels were slightly lower in primary breast tumors or non-visceral disease than in metastatic lesions or visceral diseases.

TABLE 5
Descriptions of Clinical Characteristics of Patient Subgroups in FIG. 5

| Abbreviation | Patient Characteristic |
|---|---|
| ALL | Biomarker All Patient Cohort |
| PrioChemo_NonSens. to HRi | prior chemotherapy and not sensitive to hormone |
| | therapy |
| NonSens to HRi | not sensitive to hormone therapy |
| DFI <= 24 m | disease-free intervals that were less than or equal to 24 |
| | months |
| Primary tumors | primary tumors were submitted for biomarker analysis |
| Non Visceral | non viseral diseases |
| PrioChemo | prior chemotherapy |
| Post Menopausal | post menopause at the study entry |
| PrioChemo_Sens. to HRi | prior chemotherapy and were sensitive to hormone |
| | therapy |
| DFI > 24 m | disease-free intervals that were more than 24 months |
| Sens. to HRi | sensitive to hormone therapy |
| Visceral. Mets | viseral metastatic diseases |
| Metastatic | metastatic tumors were submitted for biomarker |
| | analysis |
| Pre/Peri Menopausal | not post menopause at the study entry |
| Chemo Naive | no prior chemotherapy |

The data indicates that low KAT6A level is associated with poor prognosis for hormone therapy. Furthermore, the data show that low KAT6A level identified a subset of the patients with relatively greater benefit from addition of palbociclib to fulvestrant, especially in those with prior exposure to chemotherapy and/or with poor prognosis. The data supports the use of a KAT6A level (measured by KAT6A mRNA expression, KAT6A protein expression, KAT6A DNA gene amplification, or KAT6A DNA gene copy numbers) as a biomarker to select patients with low KAT6A level for treatment with a CDK4 inhibitor as a single agent or in combination with an antiestrogen, and, in particular, in patients with prior chemotherapy and/or with poor prognosis.

1. A method of selecting a subject having a cancer for treatment, comprising:
i) determining a KAT6A level from a biological sample of the cancer from the subject; and
ii) selecting the subject for treatment with one of:
a) a cyclin-dependent kinase 4 (CDK4) inhibitor; and
b) a cyclin-dependent kinase 4 (CDK4) inhibitor and an antiestrogen, based on the KAT6A level, wherein the KAT6A level is determined to be low.

2. A method of treating a cancer in a subject, comprising:
i) selecting the subject according to the method of claim 1; and
ii) administering to the selected subject an amount of
a) the cyclin-dependent kinase 4 (CDK4) inhibitor; or
b) the cyclin-dependent kinase 4 (CDK4) inhibitor and the antiestrogen, wherein the amounts are effective in treating the cancer.

3. The method of claim 1 or claim 2, wherein the CDK4 inhibitor is a CDK4 selective inhibitor or a CDK4/6 inhibitor.

4. The method of any one of claims 1 to 3, wherein the CDK4 inhibitor is a CDK4 selective inhibitor.

5. The method of claim 4, wherein the CDK4 selective inhibitor is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol, or a pharmaceutically acceptable salt thereof.

6. The method of any one of claims 1 to 3, wherein the CDK4 inhibitor is a CDK4/6 inhibitor.

7. The method of claim 6, wherein the CDK4/6 inhibitor is abemaciclib, ribociclib or palbociclib, or a pharmaceutically acceptable salt thereof.

8. The method of claim 7, wherein the CDK4/6 inhibitor is palbociclib, or a pharmaceutically acceptable salt thereof.

9. The method of any one of claims 1 to 8, wherein the antiestrogen is an aromatase inhibitor, a selective estrogen receptor degrader (SERD), or a selective estrogen receptor modulator (SERM).

10. The method of claim 9, wherein the antiestrogen is fulvestrant or letrozole.

11. The method of any one of claims 1 to 10, wherein the low KAT6A level is determined based on KAT6A mRNA expression, KAT6A protein expression, KAT6A DNA gene amplification, or KAT6A DNA gene copy numbers.

12. The method of claim 11, wherein the low KAT6A level is determined based on KAT6A mRNA expression.

13. The method of claim 12, wherein the low KAT6A level is greater than a reference value from normal tissue using RNA sequencing, nanostring technology, or quantitative PCR or RNA in situ hybridization (ISH) technology.

14. The method of claim 11, wherein the low KAT6A level is determined based on KAT6A protein expression.

15. The method of claim 14, wherein the low KAT6A level is less than or equal to a reference value from normal tissue using IHC assay.

16. The method of claim 11, wherein the low KAT6A level is determined based on KAT6A DNA gene amplification.

17. The method of claim 16, wherein the low KAT6A level is no DNA gene amplification.

18. The method of claim 11, wherein the low KAT6A level is determined based on KAT6A DNA gene copy numbers.

19. The method of any one of claims 1 to 18, wherein step i) of claim 1 is determined by:
a) an assay that measures KAT6A mRNA expression;
b) an assay that measures KAT6A protein expression;
c) measuring KAT6A DNA gene amplification; or
d) measuring KAT6A gene copy numbers.

20. The method of any one of claims 1 to 19, wherein the biological sample is blood, serum, cells, or tissue.

21. The method of any one of claims 1 to 20, wherein the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

22. The method of claim 21, wherein the cancer is breast cancer, lung cancer, prostate cancer, pancreatic cancer, or ovarian cancer.

23. The method of claim 22, wherein the cancer is breast cancer, lung cancer, or prostate cancer.

24. The method of claim 23, wherein the cancer is breast cancer.

25. The method of any one of claims 1 to 24, wherein the subject is human.
